(19) United States
(12) Patent Application Publication
(10) Pub. No.: US 2009/0088401 A1
(43) Pub. Date: Apr. 2, 2009

(54) In-situ cancer autovaccination with intratumoral stabilized dsRNA viral mimic

(76) Inventor: Andres Salazar, Washington, DC (US)

Correspondence Address: Mr. Max Stuhl Oppenheimer, P.O. Box 50, Stephenson, MD, 21153, US

(21) Appl. No.: 12/232,941

(22) Filed: Sep. 26, 2008

Related U.S. Application Data
(60) Provisional application No. 60/995,313, filed on Sep. 27, 2007.

(51) Int. Cl. A61K 31/713; A61P 35/04

(52) U.S. Cl. 514/44

(57) ABSTRACT

An improved autovaccination method designed to prevent or treat various neoplastic diseases by inducing a systemic immune response against a tumor and its remote metastases, consisting of the induction of an immunogenic cell death in one or more targeted tumor sites with local radiation therapy, cryotherapy, heat, chemotherapy or various other treatments, followed by intratumoral/peritumoral injection of dsRNAs (poly-ICLC in particular) in the same tumor site.

### PRIORITY

This application claims priority from provisional application 60/995,313 filed 27 Sep. 2007

### FIELD AND BACKGROUND OF THE INVENTION

1. Field of the Invention

The present invention relates in general to methods for administration of pharmaceutical compounds, and more particularly to double-stranded ribonucleic acids (dsRNA), and more particularly to polyriboinosinic-polyribocytidylic acid stabilized with polylysine and carboxymethylcellulose (Poly-ICLC).

2. Background Information

The invention described and claimed herein comprises an improved method for the adjuvant and immunomodulatory use of dsRNAs and poly-ICLC in particular, alone or in conjunction with various vaccines, radiation, chemotherapy and other treatments designed to prevent or treat various microbial, viral, neoplastic, autoimmune diseases and or degenerative diseases. DsRNAs are not normally found in mammalian cells, but are components of many viruses or byproducts of viral replication As a result, they are identified as “foreign” or as pathogen associated molecular patterns (PAMPs) by mammalian host defense systems and are potent activators of the immediate innate immune response as well as of longer-term adaptive immunity, in some ways serving as a bridge between the two systems. Poly-ICLC is a stabilized synthetic dsRNA viral mimic. Therapies using dsRNAs have yet to be approved by the US Food and Drug Administration (FDA) for any clinical indication.

The current application is aimed at a particular use of poly-ICLC as a means for inducing an effective antitumor immune response in cancer patients. U.S. Pat. No. 4,349,538 (Hilton B Levy) and patent applications US 200610223742 A1 (Andres M. Salazar) and 20040005998 A1 (Andres M. Salazar) are incorporated herein by reference.

Attempts to induce a protective immune defense against disease have captured the medical imagination for centuries, from the practice of variolation to prevent smallpox over a thousand years ago in China, to use of modem dendritic cell, DNA, peptide, and vector-based vaccines. Yet even these remain imperfect tools for inducing an effective immune response against cancers. As with viral infection, release of multiple ‘foreign’ tumor antigens induced by radiation, chemotherapy or other agents might be expected to generate an immune response against the tumor. Yet the opposite occurs, and the immune system becomes ‘tolerized’ to these tumor antigens. Multiple factors play a role in this immune failure, including antigenic shift in tumors related to their rapid growth and mutation. In addition many of the immune mechanisms that have evolved to protect the body from disease are in turn subject to inhibition or evasion by various pathogens and tumors. For example, dendritic cells (DC) play a critical early role in immunity by processing and cross-presenting foreign antigens to T-cells and other immune cells; and they identify certain specific pathogen types through a complex set of pattern recognition receptors, including the various Toll-like receptors (TLR). Yet DC and the mechanisms identifying these antigens as a foreign threat are themselves the target of inhibition by a variety of neoplastic and viral factors. (Shurin, Shurin et al. 2001) (Kaufman and Disis 2004).

### SUMMARY OF THE INVENTION

While most cancer vaccines have generally been designed to utilize one or more known antigens associated with a particular tumor type, an alternative strategy is in-situ ‘autovaccination’ or the use of the tumor itself as the antigen source, in vivo (Furumoto, Soares et al. 2003). The goal is to induce a general immune response against the primary cancer and all its (presumably identical) metastases by targeting just one accessible tumor site for treatment.

It also appears that some diseases, including Ebola virus, prostate cancer or influenza-induced anomalous DC maturation, can be reversed with Poly-ICLC, probably by virtue of its induction of a ‘natural mix’ of interferons, cytokines and chemokines, and its activation of myeloid DC via TLR3. When dosed appropriately, Poly-ICLC may supply the continual dsRNA ‘danger signal’ for an effective immune response that is usually provided by a replicating virus but that is absent in most cancers. This is in contrast to ligands such as CpG, which stimulate different TLRs and DC types targeted at different pathogens and that may not be as well suited to an antitumor cellular response. CpG is also being tested in a similar paradigm. (Brody J 2008)

Method Concept

The current invention is based on the hypothesis that intra or peritumoral administration of a TLR3 ligand such as Poly-ICLC, timed to coincide with tumor antigen release, will reverse local DC inhibition, will increase the efficiency of multiple antigen presentation to cytotoxic lymphocytes (CTL), and will prevent tolerization of tumor antigen specific CTL as well as enhancing memory T cells. (Zhu, Nishimura et al. 2007), (Salem, El-Naggar et al. 2006), (van der Most, Currie et al. 2006) Antigen release can be accomplished with low dose focal radiation, focal cryotherapy, arterial embolization, chemotherapy, or other means although it is preferable to induce an immunogenic cell death rather than physiologic cell death (Zitvogel, Apetoh et al. 2008). It is also important that the tumor antigens and the viral mimic Poly-ICLC be presented to the same dendritic cells at the same time, and preferable (subject to the patient's condition) to also administer poly-ICLC boosters two to three times per week. These boosters are expected to modify the tumor microenvironment, including induction of IL-15 and other cytokine and chemokine expression with enhancement of longevity of tumor antigen specific cytotoxic lymphocytes and memory cells. Similarly the booster shots of Poly-ICLC alone will enhance targeting of remote tumor metastases.

‘Preconditioning’ of the immune system with a judicious dose of immunosuppresants immediately prior to autovaccination with the disclosed method is also expected to synergize with the effect of poly-ICLC, possibly by decreasing regulatory T cells, eliminating previously tolerized immune cells, or otherwise ‘making immunologic space and reducing cytokine competition’ for a fresh tumor-directed cellular immune response. (Salem, Kadima et al. 2007) For example, cyclophosphamide 50 mg per day for two to three weeks prior to autovaccination is a suitable immunosuppressant. (Ghiringhelli, Menard et al. 2007)

The exact interplay between dsRNA, interferons, cytokines, chemokines and the cancer is not totally elucidated, but the role of dsRNAs such as Poly-ICLC may be bimodal: beginning with induction of interferons and gene expression of TLR3 and other systems such as OAS, PKR, RIG I, and MDA-5; and followed by their (catalytic) activation by the dsRNA. This may also underlie the natural immunologic effectiveness of viral replication, with its repeated presentation of dsRNA species to the host. In practice, double dosing with Poly-ICLC at a 24-48 hour intervals markedly boosts antiviral activity; this is also the successful dosing regimen (20 mcg/kg IM two to three times weekly) that we have used in our clinical glioma studies. (Salazar, Levy et al. 1996)

In addition to its theoretical applicability to a wide variety of cancers, potential advantages of this approach include its relative simplicity, ease, patient tolerance, and very low cost, as well as its ready testability. In collaboration with investigators at various academic institutions, Oncovir, Inc is currently testing this approach by using intratumoral Poly-ICLC combined with radiation or cryotherapy in patients with hepatoma, metastatic pancreatic cancer, lymphoma, and metastatic melanoma

### DESCRIPTION OF THE PREFERRED EMBODIMENT

Disclosed here is a method of treatment of human or veterinary neoplastic disease selected from among the group consisting of malignant brain tumors, melanoma, breast and lung cancer, colon cancer, sarcomas, renal cell cancer, leukemias, lymphomas, and other neoplasms, comprising the steps of: 1) administering to a primary or metastatic tumor site a suitable local tumoricidal agent such as radiation, chemotherapy, cryotherapy, embolization, or other agents resulting in release of tumor associated antigens, and 2) administering dsRNA intratumorally or peritunorally in two or more doses spaced 4-72 hours apart, where the dose is in a moderate range (preferably 5 to 100 mcg/kg in humans) sufficient to induce measurable but not excessive levels of serum interferon and certain other cytokines and chemokines; and for unblocking and stimulation of certain interferon and dsRNA inducible enzyme systems, Toll like receptor 3, dendritic cells, antigen specific cytotoxic lymphocytes, memory T cells and other immune cells.

Human or veterinary diseases having a tumor site may be treated, according to the disclosed invention by administering to a primary or metastatic tumor site a suitable local tumoricidal agent and administering a double stranded RNA molecule intratumorally or peritumorally. The double stranded RNA molecule is preferably selected from the group consisting of Poly-ICLC, Poly-IC (poly inosinic-polycytidilic acid), Poly-AU (poly adenylic-poly uridylic acid), or dsRNA molecules with base modifications or modifications to the nucleic acid backbone, sugar moiety, or other sites in one or both strands of the nucleic acids, or which are incorporated in liposomes or polymers, and which bind to and/or activate immune cells through an interaction with the double stranded RNA pattern recognition receptors (PRR), including but not limited to Toll-Like Receptor 3.

Diseases which should respond to the treatment include malignant brain tumors, melanoma, breast and lung cancer, colon cancer, sarcomas, renal cell cancer, hepatoma, lymphomas, and other solid neoplasms. Suitable tumoricidal agents include ionizing radiation, chemotherapy, cryotherapy, heat, embolization, or other agents resulting in release of tumor associated antigens.

The invention will be described using the example of the double stranded RNA molecule Poly-ICLC.

While dosages, dose cycles and number of repetitions are determined by the specific patient according to methods known to those of skill in the art, it is expected that a suitable treatment would include intratumoral poly-ICLC administered in a dose cycle comprising two or more doses spaced 4-72 hours apart, where the dose is in a moderate range (5 to 100 mcg/kg in humans) sufficient to induce measurable but not excessive levels of serum interferon and certain other cytokines and chemokines; and for unblocking and stimulation of certain interferon and dsRNA inducible enzyme systems, Toll like receptor 3, dendritic cells, antigen specific cytotoxic lymphocytes, memory T cells and other immune cells.

The dose cycles might be repeated weekly or twice weekly for up to one year.

An alternate regime might be chemotherapy administered for 1 day to 3 weeks prior to intratumoral poly-ICLC injection in order to decrease T regulatory cells and enhance the tumor-specific immune response. One suitable chemotherapeutic agent is cyclophosphamide, although the choice of specific agents would be patient specific and selected by methods known to those of skill in the art.

An objective is to administer in a dose which is sufficient to activate the component immune cell or cells that leads to a return of the immune response to a normal disinhibited state or to an activated state, including but not limited to dendritic cells, cytotoxic T-cells, T-cells, or B-cells; typically, this would include a dose of from 1 to 100 micrograms per kilogram of body weight, with a preferred dose of from 5 to 50 micrograms per kilogram of body weight.

Whether the quality of the cellular immune response is restored may be determined by measures such as generation of polyfunctional T-cells capable of a balanced cytokine and gamma interferon secretion.

As alternatives, Poly-ICLC may be dosed alone, prior to its subsequent dosing in combination with autovaccination, one to several times or in combination multiple times, with the dosing regimen encompassing from one week to several years, or Poly-ICLC may be dosed alone, prior to its subsequent dosing in combination with a vaccine, one to several times or in combination with a vaccine multiple times in from one to multiple cycles that span a dosing regimen that encompasses at least one month. Poly-ICLC may be administered two to three times per week for a period extending from two days to four weeks prior to presentation of antigen or vaccine.

In addition, poly-ICLC may be combined with other Pathogen Associated Molecular Patterns (PAMPs).

In addition to stimulation of dendritic cells and tumor specific cytotoxic T cells (CTL), poly-ICLC also induces interleukin-15 and other cytokines and chemokines that are important in the maintenance of specific CTL and immune memory cells, as well as in targeting of the remote tumor metastases. A closely related goal of the therapy is to improve the quality as well as the quantity of the immune response, including the generation of polyfunctional T-cells capable of a balanced cytokine and gamma interferon secretion. (Pantaleo and Koup 2004) It is thus advantageous to follow the initial induction with subsequent poly-ICLC treatments twice weekly for several weeks. In addition, repeated boosting ‘autovaccination’ cycles as described above every 4 to 8 weeks may be necessary to maintain tumor immunity.

Preconditioning with certain chemotherapeutic agents, including temozolomide and cyclophosphamide can paradoxically enhance the immune response to the autovaccination therapy.

Finally, this method could be extended by using other double-stranded RNA molecules, including but not limited to Poly-ICLC, Poly-IC, Poly-AU, dsRNA molecules with base modifications or modifications to the nucleic acid backbone, sugar moiety, or other sites in one or both strands of the nucleic acids, or encased in liposomes or various polymers, and which bind to and/or activate immune cells through an interaction with the double stranded RNA pattern recognition receptors (PRR), including but not limited to Toll-Like Receptor 3. Combination of poly-ICLC with other Pathogen Associated Molecular Patterns (PAMPs) that activate other toll-like receptors has also been reported to boost the response to certain tumors or pathogens.

### EXAMPLES

### Example A

### Clinical Protocol: Intratumoral Poly-ICLC Plus Cryotherapy in Metastatic Melanoma: A Phase I/II Study

Patients with advanced metastatic melanoma with a Karnofsdy performance score of 60 or better (able to live at home and care for most personal needs) will be selected for inclusion. A single superficial metastatic lesion will be identified for targeting. Patients will first receive a single treatment with 2-3 mg/kg of cyclophosphamide preconditioning on day 1. On day 2 the one targeted lesion will be frozen with liquid nitrogen in order to enhance tumor antigen release. This will be followed on days 2 and 4 with intratumoral/peritumoral injection of poly-ICLC (0.5 to 1 mg) Additional Poly-ICLC will then be dosed, peritumorally in paired treatments two days apart on weeks 2, 3, and 4. This will give a total of 8 poly-ICLC treatments per cycle. At the end of week four (and of week 9 and 14) there will be a one-week rest period during which patients will not receive any study medication. Each additional cycle will thus begin after a one-week rest period, and the three treatment cycles are expected to take a total of 15 weeks.

### Typical Treatment Cycle:

| Day 1 | Day 2 | Day 4 | Wk 2 | Wk 3 | Wk 4 | Wk 5 |
|---|---|---|---|---|---|---|
| Cyclophosphamide | Cryotherapy | PICLC | PICLC × 2 | PICLC × | PICLC × | Rest |
| | I.T. PICLC | | | 2 | 2 | |

Depending on patient tolerance and tumor response, additional cycles may be offered to the patient. Primary outcome measures will be tumor response in remote metastases, and survival.

### Example B

### Clinical Protocol: Autologous Vaccination Against Hepatic Cancers with Radiotherapy and Intratumoral Poly-ICLC

Hepatocellular carcinomas (HCC) produce factors blocking the host immune response and promoting immunologic tolerance to tumor antigens. Intratumoral Poly-ICLC enhances dendritic cell maturation and tumor antigen uptake, generation of specific cytotoxic lymphocytes, and targeting of tumor cells through alteration of the local tumor microenvironment, including various chemokines.

We report preliminary data from an ongoing phase I/II clinical trial (de la Torre, Salazar, et al.) using: 1) low dose 3D conformal radiation (3DRT) to increase HCC tumor antigen release, 2) Ultrasound guided intra/peri tumor injection of poly-ICLC, (Poly-ICLC) to initiate an innate and adaptive immune response in the local tumor environment, 3) percutaneous arterial embolization of the targeted lesion, and 4) systemic IM poly-ICLC to enhance immunologic memory and targeting

Patients received 3 vaccination cycles over 3-4 months as follows:
Day 1, 2, 3: 2.5 gray 3DRT.
Day 4: ultrasound guided peri/intra-tumoral poly-ICLC (0.25 to 1 mg in 5 cc saline) & embolization;
twice weekly for three weeks: Intramuscular poly-ICLC (20 mcg/kg)

### Preliminary Results:

| | | Total Diam | | | |
|---|---|---|---|---|---|
| Patient | BCLC | (cm) | Total Lesions | Toxicity | Tumor status |
| 1 | C | 17 | 4 | LF, D | N/A |
| 2 | C | 8 | 3 | N, F, Fev | Stable at 6 months |
| 3 | B | 15 | 4 | A, Fev | PR at 6 months |
| 4 | B | 8 | 1 | N, A, DH, Fev | Stable at 4 months |
| 5 | B | 7 | 5 | F, A, Fev | PR at 3 months |

BCLC: Barcelona Clinic Liver Cancer scale,
LF: liver failure,
D: death,
N: nausea,
F: fatigue,
Fev: fever,
DH: dehydration,
A: anorexia,
PR: partial response

Patients have tolerated the treatment without significant side effects and remain clinically well. Currently, no new lesions have been detected on follow-up triple phase CT in the 4 patients completing the protocol. Two patients have shown radiologic response in remote lesions that were not originally targeted by radiation, indicating a systemic immune response that is capable of targeting remote metastases. These early data show that tumor conditioning with radiation, intra-tumoral injection of poly-ICLC TLR3 ligand into liver cancers, followed by tumor arterial embolization is feasible and safe in humans. Stabilization and/or response in these typically very aggressive tumors is particularly promising and further supports the concept behind this disclosure.

### REFERENCES CITED

Brody J, A. W., Cerwinski D, Advani R, Horning S J, Ganjoo R, Levy R (2008). “Clinical and immonologic responses to a novel in situ lymphoma vaccine maneuver: Preliminary results of a phase II trial of intratumoral CpG 7909.” J Clin Oncology 26(May 20 suppl, abstr 3003).
Furumoto, K., L. Soares, et al. (2003). “Induction of potent antitumor immunity by in situ targeting of intratumoral DCs.” J Clin Investig 113(5): 774-783.
Ghiringhelli, F., C. Menard, et al. (2007). “Metronomic cyclophosphamide regime selectively depletes CD4+CD25+regulatory T cells and restorers T and KN effector function in end stage cancer patients.” Cancer Immumol Immunother 56(5): 641-648.
Kaufman, H. and M. Disis (2004). “Immune system versus tumor: shifting the balance in favor of DCs and effective immunity.” J Clin Invest 113(5): 664-667.
Pantaleo, G. and R. Koup (2004). “Correlates of immune protection in HIV-1 infection: what we know, what we don't know, what we should know.” Nature Medicine 10(8): 806-810.
Salazar, A., H. Levy, et al. (1996). “Long-term IM Poly-ICLC treatment of malignant glioma: an open pilot study.” Neurosurgery 38(6): 1096-1104.
Salem, M., S. El-Naggar, et al. (2006). “The adjuvant effects of the toll-like receptor-3 ligand polyinosinic-cytidylic acid poly (I:C) on antigen specific CD8=T cell responses are partially dependent on NK cells with the induction of a beneficial cytokine milieu.” Vaccine 24(24): 5119-32.
Salem, M., A. Kadima, et al. (2007). “Defining the ability of cyclophosphamide preconditioning to enhance the antigen specific CD8+Tcell response to peptide vaccination: creation of a beneficial host microenvironment involving type 1 IFNs and Myeloid cells.” J Immunother.
Shurin, G., M. Shurin et al. (2001). “Neuroblastoma-derived gangliosides inhibit dendritic cell generation and function.” Cancer Research 61: 363-369.
van der Most, R., A. Currie, et al. (2006). “Cranking the immunologic engine with chemotherapy: using context to drive tumor antigen cross-presentation towards antitumor immunity.” Cancer Res 66(2): 60-604.
Zhu, X., F. Nishimura, et al. (2007). “Toll lik receptor 3 ligand Poly-ICLC promotes the efficacy of peripheral vaccinations with tumor antigen-derived peptide epitopes in murine CNS tumor models.” J Translational Med 5(10): 1-15.
Zitvogel, L., L. Apetoh, et al. (2008). “The anticancer immune response: indispensable for therapeutic success?” J Clin Investig 118(6): 1991-2001.

I claim:

1. A method of treatment of human or veterinary disease having a tumor site, comprising the steps of: 1) administering to a primary or metastatic tumor site a suitable local tumoricidal agent and 2) administering a double stranded RNA molecule intratumorally or peritumorally.

2. The method of claim 1 wherein said double stranded RNA molecule is selected from the group consisting of Poly-ICLC, Poly-IC, Poly-AU, dsRNA molecules with base modifications or modifications to the nucleic acid backbone, sugar moiety, or other sites in one or both strands of the nucleic acids, or which are incorporated in liposomes or polymers, and which bind to and/or activate immune cells through an interaction with the double stranded RNA pattern recognition receptors (PRR), including but not limited to Toll-Like Receptor 3.

3. The method of claim 1 wherein said double stranded RNA molecule is Poly-ICLC.

4. The method of claim 3 wherein said human or veterinary disease is selected from among the group consisting of malignant brain tumors, melanoma, breast and lung cancer, colon cancer, sarcomas, renal cell cancer, hepatomas, lymphomas, and other solid neoplasms.

5. The method of claim 3 wherein the tumoricidal agent is ionizing radiation, chemotherapy, cryotherapy, heat, embolization, or other agents resulting in release of tumor associated antigens.

6. The method of claim 3 wherein intratumoral poly-ICLC is administered in a dose cycle comprising two or more doses spaced 4-72 hours apart, where the dose is sufficient to induce measurable but not excessive levels of serum interferon and other cytokines and chemokines; and for unblocking and stimulation of certain interferon and dsRNA inducible enzyme systems, Toll like receptor 3, dendritic cells, antigen specific cytotoxic lymphocytes, memory T cells and other immune cells.

7. The method of claim 3 wherein the dose cycles are repeated weekly or twice weekly for up to one year.

8. The method of claim 3 wherein chemotherapy is administered for 1 day to 3 weeks prior to intratumoral poly-ICLC injection in order to decrease T regulatory cells and enhance the tumor-specific immune response.

9. The method of claim 3 wherein the chemotherapeutic agent is cyclophosphamide.

10. The method of claim 3 wherein poly-ICLC is administered in a dose which is sufficient to activate the component immune cell or cells that leads to a return of the immune response to a normal disinhibited state or to an activated state, including but not limited to dendritic cells, cytotoxic T-cells, T-cells, or B-cells; at a dose of from 1 to 100 micrograms per kilogram of body weight.

11. The method of claim 3 where the preferred dose of Poly-ICLC is from 5 to 50 micrograms per kilogram of body weight.

12. The method of claim 10 wherein the quality of the cellular immune response is restored, such as generation of polyfunctional T-cells capable of a balanced cytokine and gamma interferon secretion.

13. The method of claim 3 wherein Poly-ICLC is dosed alone, prior to its subsequent dosing in combination with autovaccination, one to several times or in combination multiple times.

14. The method of claim 13 wherein multiple cycles are administered in a dosing regimen that encompasses from one week to several years.

15. The method of claim 3, wherein the Poly-ICLC is dosed alone, prior to its subsequent dosing in combination with a vaccine, one to several times or in combination with a vaccine multiple times in from one to multiple cycles that span a dosing regimen that encompasses at least one month.

16. The method of claim 3 wherein Poly-ICLC is administered two to three times per week for a period extending from two days to four weeks prior to presentation of antigen or vaccine.

17. The method of claim 3 wherein poly-ICLC is combined with other Pathogen Associated Molecular Patterns (PAMPs).
